(19) United States
(12) Patent Application Publication
(10) Pub. No.: US 2006/0084590 A1
(43) Pub. Date: Apr. 20, 2006

(54) Methods of and compounds for modulating the activity of bacterial FabG

(76) Inventors: Thomas D. Meek, King of Prussia, PA (US); Mehul Patel, King of Prussia, PA (US); Sara H. Thrall, King of Prussia, PA (US)

Correspondence Address: Edward R. Gimmi;SmithKline Beecham Corporation, Corporate Intellectual Property-U.S., UW2220, P.O. Box 1539, King of Prussia, PA, 19406-0939, US

(21) Appl. No.: 10/024,808

(22) Filed: Dec. 19, 2001

Related U.S. Application Data
(60) Provisional application No. 60/259,595, filed on Jan. 3, 2001.

(51) Int. Cl. A61K 38/16; A61K 31/00

(52) U.S. Cl. 514/1; 514/12

(57) ABSTRACT

Prokaryotic FAB G polypeptides and DNA (RNA) encoding such FAB G and a procedure for producing such polypeptides by recombinant techniques is disclosed. Also disclosed are methods for utilizing such FAB G for the treatment of infection, such as bacterial infections. Antagonists against such FAB G and their use as a therapeutic to treat infections, such as staphylococcal infections are also disclosed. Also disclosed are diagnostic assays for detecting diseases related to the presence of FAB G nucleic acid sequences and the polypeptides in a host. Also disclosed are diagnostic assays for detecting polynucleotides encoding FAB G and for detecting the polypeptide in a host.

### FIELD OF THE INVENTION

This invention relates to antagonists against FAB G polypeptide and their use as a therapeutic to treat infections, such as staphylococcal infections, which are also disclosed. Further disclosed are methods of treating disease using a compound to agonize or antagonize a mechanism of action of activity of Fab G.

### BACKGROUND OF THE INVENTION

Fatty acid biosynthesis is essential for the production of structural components of bacterial membranes. Streptococcus pneumoniae FabG catalyzes the NADPH-dependent reduction of acetoacetyl-acyl carrier protein (herin “ACP”) to generate β-hydroxyacyl-ACP.

### SUMMARY OF THE INVENTION

Provided herein are an antagonist that inhibits or an agonist that activates an activity of a polypeptide selected from the group consisting of: a polypeptide comprising an amino acid sequence which is at least 90% identical to the amino acid sequence of SEQ ID NO:2, and a polypeptide comprising an amino acid sequence as set forth in SEQ ID NO:2.

Further provided is a method for the treatment of an individual infected with a bacteria comprising the steps of administering to the individual an antibacterially effective amount of an antagonist that inhibits or an agonist that activates an activity of Fab G. The invention also provides a method for inhibiting or activating an activity of Fab G polypeptide comprising the steps of contacting a composition comprising said polypeptide with an effective amount of an antagonist that inhibits or agonist that activates an activity of Fab G.

The invention provides an antagonist that inhibits or an agonist that activates an activity of a polypeptide selected from the group consisting of: a polypeptide comprising an amino acid sequence which is at least 90% identical to the amino acid sequence of SEQ ID NO:2, and a polypeptide comprising an amino acid sequence as set forth in SEQ ID NO:2, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation of a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

The invention also provides a method for the treatment of an individual having need to inhibit or activate Fab G polypeptide comprising the steps of: administering to the individual an antibacterially effective amount of an antagonist that inhibits or an agonist that activates an activity of a polypeptide selected from the group consisting of: a polypeptide comprising an amino acid sequence which is at least 90% identical to the amino acid sequence of SEQ ID NO:2, and a polypeptide comprising an amino acid sequence as set forth in SEQ ID NO:2, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation of a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

The invention still further provides a method for the treatment of an individual infected with a bacteria comprising the steps of administering to the individual an antibacterially effective amount of an antagonist that inhibits or an agonist that activates an activity of a polypeptide selected from the group consisting of: a polypeptide comprising an amino acid sequence which is at least 90% identical to the amino acid sequence of SEQ ID NO:2, and a polypeptide comprising an amino acid sequence as set forth in SEQ ID NO:2, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

Also provided by the invention is a method wherein said bacteria is selected from the group consisting of a member of the genus Staphylococcus, Staphylococcus aureus, a member of the genus Streptococcus, and Streptococcus pneumoniae.

Further provided by the invention is a method for the treatment of an individual having need to inhibit or activate Fab G polypeptide comprising the steps of administering to the individual an antibacterially effective amount of an antagonist that inhibits or an agonist that activates an activity of Fab G selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

The invention provides a method for the treatment of an individual infected with a bacteria comprising the steps of administering to the individual an antibacterially effective amount of an antagonist that inhibits or an agonist that activates that activates an activity of Fab G selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

The invention provides another method wherein said bacteria is selected from the group consisting of: a member of the genus Staphylococcus, Staphylococcus aureus, a member of the genus Streptococcus, and Streptococcus pneumoniae.

A further method is provided for the treatment of an individual infected by Streptococcus pneumoniae comprising the steps of administering to the individual an antibacterially effective amount of an antagonist that inhibits or antagonist that activates an activity of Streptococcus pneumoniae Fab G selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the βhydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

The invention provides an antagonist that inhibits an activity of a polypeptide selected from the group consisting of: a polypeptide comprising an amino acid sequence which is at least 90% identical to the amino acid sequence of SEQ ID NO:1, and a polypeptide comprising an amino acid sequence as set forth in SEQ ID NO:1, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

Also provided by the invention is a method for the treatment of an individual having need to inhibit Fab G polypeptide comprising the steps of administering to the individual an antibacterially effective amount of an antagonist that inhibits an activity of a polypeptide selected from the group consisting of: a polypeptide comprising an amino acid sequence which is at least 90% identical to the amino acid sequence of SEQ ID NO:1, and a polypeptide comprising an amino acid sequence as set forth in SEQ ID NO:1, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

Another method of the invention provides a method for inhibiting an activity of Fab G polypeptide comprising the steps of contacting a composition comprising said polypeptide with an effective amount of an antagonist that inhibits an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

The invention also provides a method for inhibiting an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

Still further provided is a method wherein said bacteria is selected from the group consisting of: a member of the genus Staphylococcus, Staphylococcus aureus, a member of the genus Streptococcus, and Streptococcus pneumoniae.

A method is also provided for inhibiting a growth of bacteria comprising the steps of contacting a composition comprising bacteria with an antibacterially effective amount of an antagonist that inhibits an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

A method is also provide wherein said bacteria is selected from the group consisting of:

a member of the genus Staphylococcus, Staphylococcus aureus, a member of the genus Streptococcus, and Streptococcus pneumoniae.

A method for inhibiting a Fab G polypeptide comprising the steps of contacting a composition comprising bacteria with an antibacterially effective amount of an antagonist that inhibits an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue (FIG. 5); formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

A method is also provided wherein said bacteria is selected from the group consisting of:

a member of the genus Staphylococcus, Staphylococcus aureus, a member of the genus Streptococcus, and Streptococcus pneumoniae.

### BRIEF DESCRIPTION OF THE DRAWINGS

FIG. 1A shows a plot of log k_{cat} vs. pH characterized by a “half-bell” curve. FIGS. 1B and 1C, repectively, show plots of log k_{cat}/K_{AcAc-ACP} vs. pH and log k_{cat}/K_{NADPH} vs. pH

FIG. 2 shows primary deuterium (FIG. 2A) and solvent kinetic isotope effects (FIG. 2B).

FIG. 3 shows an inverse solvent kinetic isotope effect on k_{cat}.

FIG. 4 shows models based on homologous systems presented to account for the substrate induced change in the solvent isotope effect.

FIG. 5 shows a chemical mechanism of the reaction which is stepwise and in which hydride transfer from NADPH proceeds proton transfer from the Lys residue.

### DESCRIPTION OF THE INVENTION

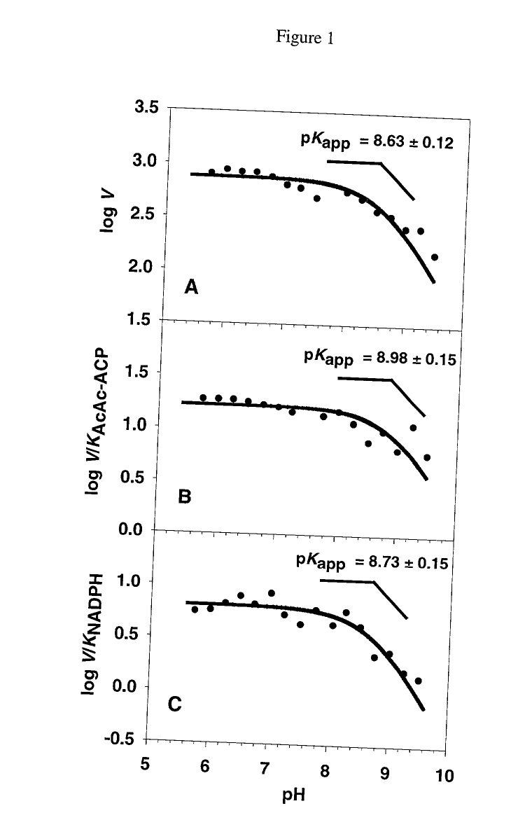

A number of kinetic parameters have been resolved for FabG and are provided herein as mechanistic targets for methods of treating disease and modulating activities of certain organisms, partcularly pathogens. These embodiments are set forth in more detail herein. Mechanistic enzymology of FabG made possible certasin of these embodiments. For instance, pH dependence of kinetic parameters, k_{cat} and k_{cat}/Kₘ for both NADPH and acetoacetyl-ACP (AcAc-ACP) have been determined for FabG. A plot of log k_{cat} vs. pH is characterized by a “half-bell” curve in which deprotonation of a group with a pK of 8.6±0.1 leads to a diminution in k_{cat} (FIG. 1A). This group is indicated to be enzymatic, and likely serves as the general acid responsible for donating a proton to the carbonyl oxygen during its reduction. Similarly, plots of log k_{cat}/K_{AcAc-ACP} vs. pH (FIG. 1B) and log k_{cat}/K_{NADPH} vs. pH (FIG. 1C) also decrease at high pH as two distinct groups are deprotonated, with apparent pK values of 9.0±0.2 and 8.7±0.2, respectively. The ionizable group in the plot of log k_{cat}/K_{NADPH} vs. pH profile putatively binds the pyrophosphate bridge of NADPH. The group arising from the k_{cat}/K_{AcAc-ACP} and k_{cat} plots are indicated to be the same residue implicated in affecting catalysis. However, this group may also be a lysine residue involved as a general acid in the catalysis.

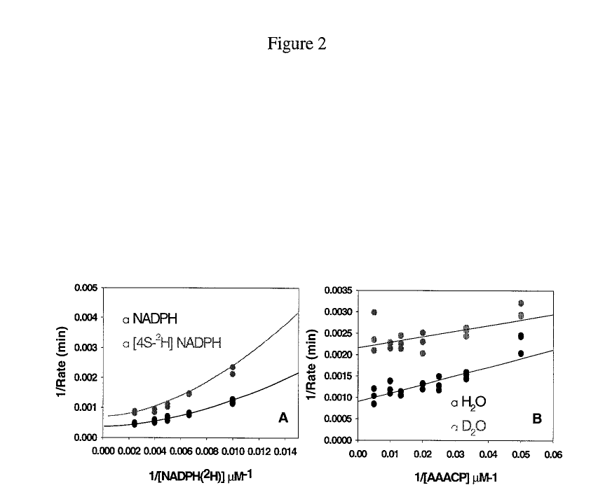

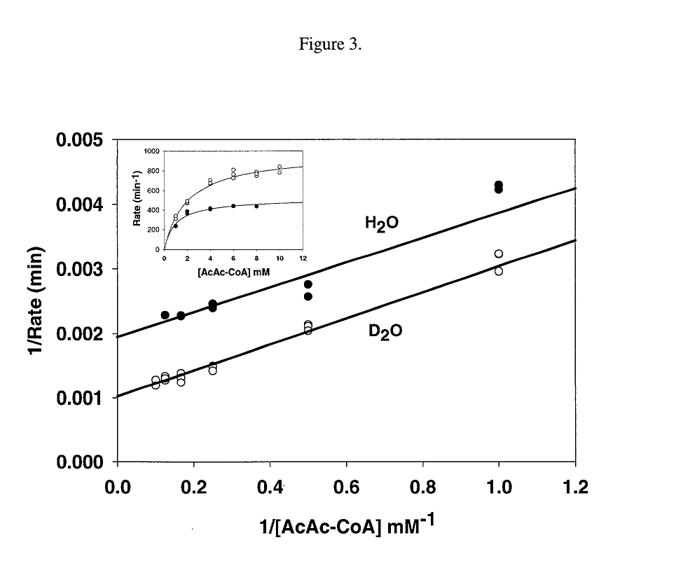

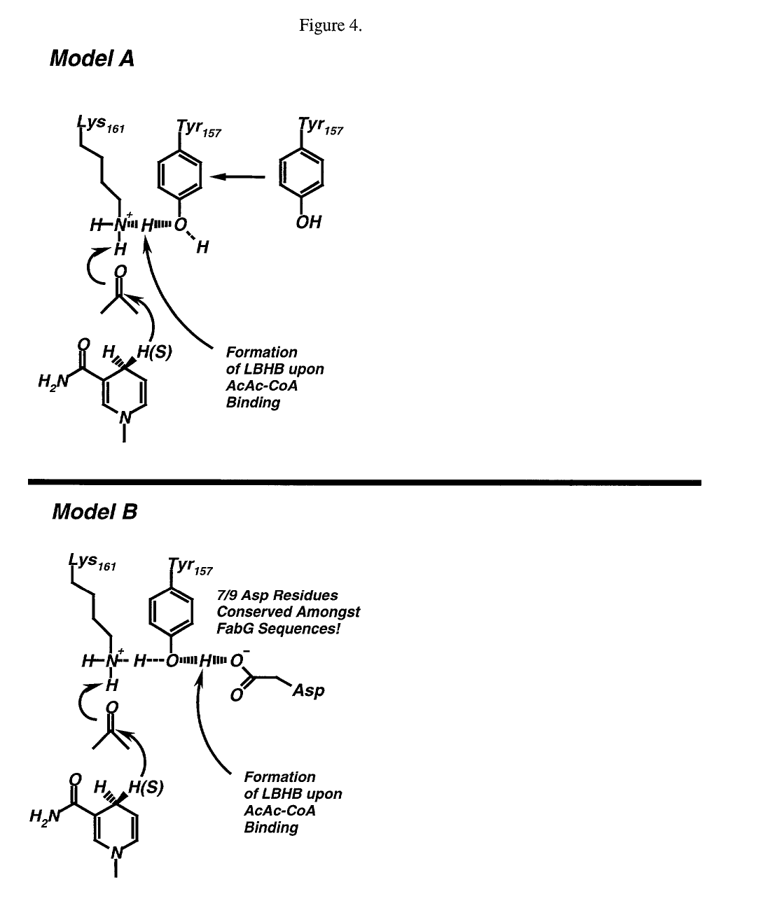

The primary deuterium (FIG. 2A) and solvent kinetic isotope effects (FIG. 2B) have been determined with values of ^{D}k_{cat(H}_{₂}_{O)}=1.9±0.2 and ^{D}k_{cat(H}_{₂}_{O)}/K_{NADPH}=1.5±0.4 and ^{D}^{₂}^{O}k_{cat}=2.1±0.1 and ^{D}^{₂}^{O}k_{cat}/K_{AcAc-ACP}=0.6±0.3, respectively. An inverse solvent kinetic isotope effect on k_{cat} (^{D}^{₂}^{O}k_{cat}=0.52±0.03, ^{D}^{₂}^{O}k_{cat}/K_{AcAc-CoA}=1.05±0.19) (FIG. 3) with the truncated acyl substrate, acetoacetyl-CoA, indicates a subtle conformational change which is believed to induce formation a low-barrier hydrogen bond (LBHB) in the mechanism of ketone reduction. Two embodiments of the invention are models based on homologous systems presented to account for the substrate induced change in the solvent isotope effect (FIG. 4). In model A, a mechanism based on 3-α-hydroxysteroid dehydrogenase (Schlegel, B. P. et al (1998) Biochemistry 37, 3538-3548), a conformational change upon acetoacetyl-CoA binding is believed to result in the formation of a LBHB between Tyr157 and Lys161. The resultant energy from forming a LBHB between Tyr157 and Lys161 may facilitate the proton transfer from Lys157 to the carbonyl oxygen. This mechanism of active site “compression” in model B is similar to model A. However, in model B the LBHB is forming between Tyr157 and an Asp residue, similar to that seen for chymotrypsin (Cassidy, C. S. et al (2000) BBRC 23, 789-792). In this model, the formation of a LBHB between an Asp residue and Tyr157 would strengthen the role of Tyr157 in facilitating general acid catalysis by Lys161. Both models portray compression of the active site resulting in the formation of a LBHB that ultimately facilitates proton transfer to the carbonyl oxygen.

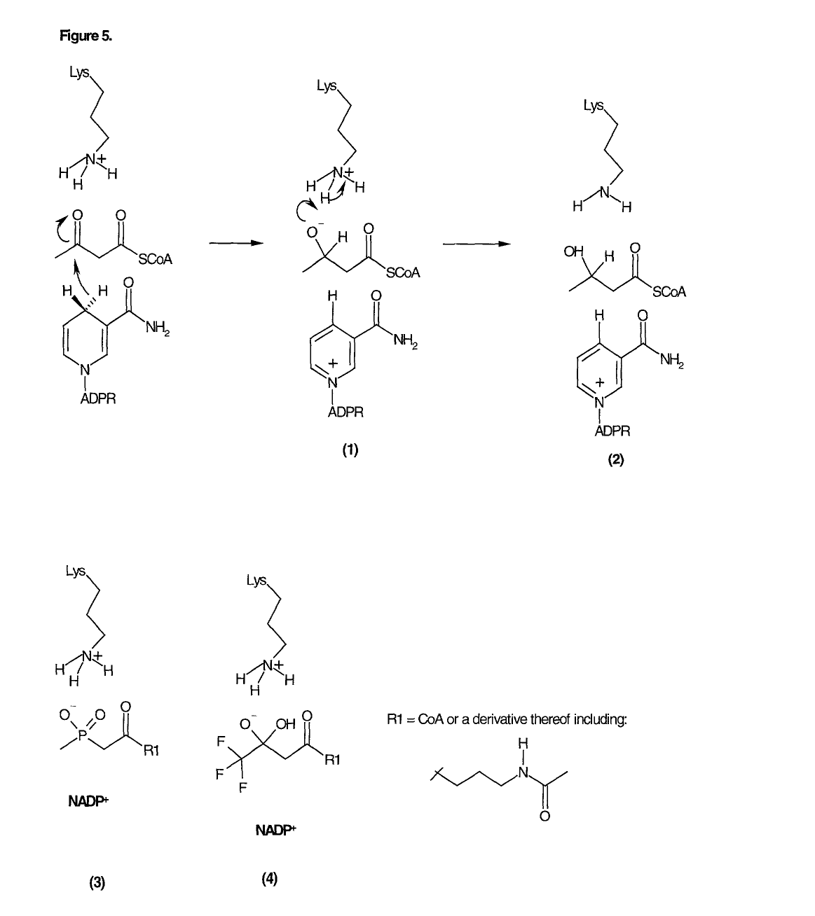

An embodiment of the invention is a chemical mechanism of the reaction which is stepwise and in which hydride transfer from NADPH proceeds proton transfer from the Lys residue (FIG. 5), the anionic, tetrahedral reaction intermediate (1) is likely to be formed, and is potentially charge-stabilized by the protonated Lys group prior to proton transfer to form the βhydroxy-keto product (2). A further embodiment is a chemical mechanism and, thus, a β-phosphino-keto derivative 3 or a β-trifluoroketo-keto derivative 4 of AcAc-CoA represent rationally-derived inhibitors for FabG that structurally mimic the reaction intermediate (1) in that the tetrahedral anionic structures of these derivatives would form ionic pairs with the protonated Lys. In these structures, R1 could represent either coenzyme A or a structural derivative thereof, such as pantheinate or a derivative thereof.

Use of a phosphinate to mimic a reaction intermediate such as 1 for another NADPH-dependent reductase has a precedent (Dreyer, G. B., Garvie, C. T., Metcalf, B. W., Meek, T. D. and Mayer, R. J. (1991) “Phosphinic Acid Inhibitors of 3-Hydroxy-3-Methylglutaryl Coenzyme A Reductase” Bioorg. Med. Chem. Lett. 1, 151-154).

Each of the enzymatic steps provided herein, particularly in the forgoing provide targets for compounds useful for, but not limited to the treatment of disease, particularly diseases caused by or related to organisms, especially pathogens. Moreover, these enzymatic steps provide targets for compounds useful for decontamination or deifestation or materials and compounds by organisms, particularly those that cause or are related to disease.

TABLE 1

Polynucleotide and Polypeptide Sequences
The following represent sequences useful in embodiments of the invention. The
invention is not limited to use of such sequences.

(A) Streptococcus pneumoniae FabG
polynucleotide sequence.

[SEQ ID NO:1]

5′-ATGAAACTAGAACATAAAAATATCTTTATTACAGGTTCGAGTCGTGG
AATTGGTCTTGCCATCGCCCACAAGTTTGCTCAAGCAGGAGCCAACATTG
TCTTAAACAGTCGTGGGGCAATCTCAGAAGAATTGCTCGCTGAGTTTTCA
AACTATGGTATCAAGGTGGTTCCCATTTCAGGAGATGTATCAGATTTTGC
AGACGCTAAGCGTATGATTGATCAAGCTATTGCAGAACTGGGTTCAGTAG
ATGTTTTGGTCAACAATGCAGGGATTACCCAAGATACTCTTATGCTCAAG
ATGACAGAAGCAGATTTTGAAAAAGTGCTCAAGGTCAATCTGACTGGTGC
CTTTAATATGACACAATCAGTCTTGAAACCGATGATGAAAGCCAGAGAAG
GTGCTATCATTAATATGTCTAGTGTTGTTGGTTTGATGGGGAATATTGGT
CAAGCTAACTATGCTGCTTCTAAGGCTGGCTTGATTGGCTTTACCAAGTC
TGTGGCACGCGAGGTCGCTAGTCGGAATATACGAGTCAATGTGATTGCTC
CAGGAATGATTGAGTCTGATATGACAGCTATCTTATCAGATAAGATTAAG
GAAGCTACACTAGCTCAGATTCCGATGAAAGAATTTGGGCAGGCAGAGCA
GGTTGCAGATTTGACAGTATTTTTAGCAGGCCAAGATTATCTAACTGGTC
AAGTGATTGCCATTGATGGTGGCTTAAGTATGTAG-3′
(B) Streptococcus pneumoniae FabG polypeptide
sequence deduced from a polynucleotide
sequence in this table.

[SEQ ID NO:2]

NH₂-MKLEHKNIFITGSSRGIGLAIAHKFAQAGANIVLNSRGAISEELLA
EFSNYGIKVVPISGDVSDFADAKRMIDQAIAELGSVDVLVNNAGITQDTL
MLKMTEADFEKVLKVNLTGAFNMTQSVLKPMMKAREGAIINMSSVVGLMG
NIGQANYAASKAGLIGFTKSVAREVASRNIRVNVIAPGMIESDMTAILSD
KIKEATLAQIPMKEFGQAEQVADLTVFLAGQDYLTGQVIAIDGGLSM*-
COOH

Deposit Materials

A deposit comprising a Streptococcus pneumoniae 0100993 strain has been deposited with the National Collections of Industrial and Marine Bacteria Ltd. (herein “NCIMB”), 23 St. Machar Drive, Aberdeen AB2 1RY, Scotland on 11 Apr. 1996 and assigned deposit number 40794. The deposit was described as Streptococcus pneumoniae 0100993 on deposit.

On 17 Apr. 1996 a Streptococcus pneumoniae 0100993 DNA library E. coli was similarly deposited with the NCIMB and assigned deposit number 40800. The Streptococcus pneumoniae strain deposit is referred to herein as “the deposited strain” or as “the DNA of the deposited strain.”

The deposited strain comprises a full length FabG gene. The sequence of the polynucleotides comprised in the deposited strain, as well as the amino acid sequence of any polypeptide encoded thereby, are controlling in the event of any conflict with any description of sequences herein.

The deposit of the deposited strain has been made under the terms of the Budapest Treaty on the International Recognition of the Deposit of Micro-organisms for Purposes of Patent Procedure. The deposited strain will be irrevocably and without restriction or condition released to the public upon the issuance of a patent. The deposited strain is provided merely as convenience to those of skill in the art and is not an admission that a deposit is required for enablement, such as that required under 35 U.S.C. §112. A license may be required to make, use or sell the deposited strain, and compounds derived therefrom, and no such license is hereby granted.

In one aspect of the invention there is provided an isolated nucleic acid molecule encoding a mature polypeptide expressible by the Streptococcus pneumoniae 0100993 strain, which polypeptide is comprised in the deposited strain. Further provided by the invention are FabG polynucleotide sequences in the deposited strain, such as DNA and RNA, and amino acid sequences encoded thereby. Also provided by the invention are FabG polypeptide and polynucleotide sequences isolated from the deposited strain.

Polypeptides

FabG polypeptide of the invention is substantially phylogenetically related to other proteins of the fabG (3-oxoacyl-acyl carrier protein reductase) family.

In one aspect of the invention there are provided polypeptides of Streptococcus pneumoniae referred to herein as “FabG” and “FabG polypeptides” as well as biologically, diagnostically, prophylactically, clinically or therapeutically useful variants thereof, and compositions comprising the same.

Among the particularly preferred embodiments of the invention are variants of FabG polypeptide encoded by naturally occurring alleles of a FabG gene. The present invention further provides for an isolated polypeptide that: (a) comprises or consists of an amino acid sequence that has at least 95% identity, most preferably at least 97-99% or exact identity, to that of SEQ ID NO:2 over the entire length of SEQ ID NO:2; (b) a polypeptide encoded by an isolated polynucleotide comprising or consisting of a polynucleotide sequence that has at least 95% identity, even more preferably at least 97-99% or exact identity to SEQ ID NO:1 over the entire length of SEQ ID NO:1; (c) a polypeptide encoded by an isolated polynucleotide comprising or consisting of a polynucleotide sequence encoding a polypeptide that has at least 95% identity, even more preferably at least 97-99% or exact identity, to the amino acid sequence of SEQ ID NO:2, over the entire length of SEQ ID NO:2.

The polypeptides of the invention include a polypeptide of Table 1 [SEQ ID NO:2] (in particular a mature polypeptide) as well as polypeptides and fragments, particularly those that has a biological activity of FabG, and also those that have at least 95% identity to a polypeptide of Table 1 [SEQ ID NO:2] and also include portions of such polypeptides with such portion of the polypeptide generally comprising at least 30 amino acids and more preferably at least 50 amino acids.

The invention also includes a polypeptide consisting of or comprising a polypeptide of the formula:
X—(R₁)ₘ—(R₂)—(R₃)ₙ—Y
wherein, at the amino terminus, X is hydrogen, a metal or any other moiety described herein for modified polypeptides, and at the carboxyl terminus, Y is hydrogen, a metal or any other moiety described herein for modified polypeptides, R₁ and R₃ are any amino acid residue or modified amino acid residue, m is an integer between 1 and 1000 or zero, n is an integer between 1 and 1000 or zero, and R₂ is an amino acid sequence of the invention, particularly an amino acid sequence selected from Table 1 or modified forms thereof. In the formula above, R₂ is oriented so that its amino terminal amino acid residue is at the left, covalently bound to R₁, and its carboxy terminal amino acid residue is at the right, covalently bound to R₃. Any stretch of amino acid residues denoted by either R₁ or R₃, where m and/or n is greater than 1, may be either a heteropolymer or a homopolymer, preferably a heteropolymer. Other preferred embodiments of the invention are provided where m is an integer between 1 and 50, 100 or 500, and n is an integer between 1 and 50, 100, or 500.

It is most preferred that a polypeptide of the invention is derived from Streptococcus pneumoniae, however, it may preferably be obtained from other organisms of the same taxonomic genus. A polypeptide of the invention may also be obtained, for example, from organisms of the same taxonomic family or order.

A fragment is a variant polypeptide having an amino acid sequence that is entirely the same as part but not all of any amino acid sequence of any polypeptide of the invention. As with FabG polypeptides, fragments may be “free-standing,” or comprised within a larger polypeptide of which they form a part or region, most preferably as a single continuous region in a single larger polypeptide.

Preferred fragments include, for example, truncation polypeptides having a portion of an amino acid sequence of Table 1 [SEQ ID NO:2], or of variants thereof, such as a continuous series of residues that includes an amino- and/or carboxyl-termiinal amino acid sequence. Degradation forms of the polypeptides of the invention produced by or in a host cell, particularly a Streptococcus pneunoniae, are also preferred. Further preferred are fragments characterized by structural or functional attributes such as fragments that comprise alpha-helix and alpha-helix forming regions, beta-sheet and beta-sheet-forming regions, turn and tum-forming regions, coil and coil-forming regions, hydrophilic regions, hydrophobic regions, alpha amphipathic regions, beta amphipathic regions, flexible regions, surface-forming regions, substrate binding region, and high antigenic index regions.

Further preferred fragments include an isolated polypeptide comprising an amino acid sequence having at least 15, 20, 30, 40, 50 or 100 contiguous amino acids from the amino acid sequence of SEQ ID NO:2, or an isolated polypeptide comprising an amino acid sequence having at least 15, 20, 30, 40, 50 or 100 contiguous amino acids truncated or deleted from the amino acid sequence of SEQ ID NO:2.

Fragments of the polypeptides of the invention may be employed for producing the corresponding full-length polypeptide by peptide synthesis; therefore, these variants may be employed as intermediates for producing the full-length polypeptides of the invention.

Antagonists and Agonists—Assays and Molecules

Polypeptides and polynucleotides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

Polypeptides and polynucleotides of the present invention are responsible for many biological functions, including many disease states, in particular the Diseases herein mentioned. It is therefore desirable to devise screening methods to identify compounds that agonize (e.g., stimulate) or that antagonize (e.g.,inhibit) the function of the polypeptide or polynucleotide. Accordingly, in a further aspect, the present invention provides for a method of screening compounds to identify those that agonize or that antagonize the function of a polypeptide or polynucleotide of the invention, as well as related polypeptides and polynucleotides. In general, agonists or antagonists (e.g., inhibitors) may be employed for therapeutic and prophylactic purposes for such Diseases as herein mentioned. Compounds may be identified from a variety of sources, for example, cells, cell-free preparations, chemical libraries, and natural product mixtures. Such agonists and antagonists so-identified may be natural or modified substrates, ligands, receptors, enzymes, etc., as the case may be, of FabG polypeptides and polynucleotides; or may be structural or functional mimetics thereof (see Coligan et al., Current Protocols in Immunology 1(2):Chapter 5 (1991)).

The screening methods may simply measure the binding of a candidate compound to the polypeptide or polynucleotide, or to cells or membranes bearing the polypeptide or polynucleotide, or a fusion protein of the polypeptide by means of a label directly or indirectly associated with the candidate compound. Alternatively, the screening method may involve competition with a labeled competitor. Further, these screening methods may test whether the candidate compound results in a signal generated by activation or inhibition of the polypeptide or polynucleotide, using detection systems appropriate to the cells comprising the polypeptide or polynucleotide. Inhibitors of activation are generally assayed in the presence of a known agonist and the effect on activation by the agonist by the presence of the candidate compound is observed. Constitutively active polypeptide and/or constitutively expressed polypeptides and polynucleotides may be employed in screening methods for inverse agonists, in the absence of an agonist or antagonist, by testing whether the candidate compound results in inhibition of activation of the polypeptide or polynucleotide, as the case may be. Further, the screening methods may simply comprise the steps of mixing a candidate compound with a solution comprising a polypeptide or polynucleotide of the present invention, to form a mixture, measuring FabG polypeptide and/or polynucleotide activity in the mixture, and comparing the FabG polypeptide and/or polynucleotide activity of the mixture to a standard. Fusion proteins, such as those made from Fc portion and FabG polypeptide, as herein described, can also be used for high-throughput screening assays to identify antagonists of the polypeptide of the present invention, as well as of phylogenetically and and/or functionally related polypeptides (see D. Bennett et al., J Mol Recognition, 8:52-58 (1995); and K. Johanson et al., J Biol Chem, 270(16):9459-9471 (1995)). The polynucleotides, polypeptides and antibodies that bind to and/or interact with a polypeptide of the present invention may also be used to configure screening methods for detecting the effect of added compounds on the production of mRNA and/or polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

The invention also provides a method of screening compounds to identify those that enhance (agonist) or block (antagonist) the action of FabG polypeptides or polynucleotides, particularly those compounds that are bacteristatic and/or bactericidal. The method of screening may involve high-throughput techniques. For example, to screen for agonists or antagonists, a synthetic reaction mix, a cellular compartment, such as a membrane, cell envelope or cell wall, or a preparation of any thereof, comprising FabG polypeptide and a labeled substrate or ligand of such polypeptide is incubated in the absence or the presence of a candidate molecule that may be a FabG agonist or antagonist. The ability of the candidate molecule to agonize or antagonize the FabG polypeptide is reflected in decreased binding of the labeled ligand or decreased production of product from such substrate. Molecules that bind gratuitously, i.e., without inducing the effects of FabG polypeptide are most likely to be good antagonists. Molecules that bind well and, as the case may be, increase the rate of product production from substrate, increase signal transduction, or increase chemical channel activity are agonists. Detection of the rate or level of, as the case may be, production of product from substrate, signal transduction, or chemical channel activity may be enhanced by using a reporter system. Reporter systems that may be useful in this regard include but are not limited to calorimetric, labeled substrate converted into product, a reporter gene that is responsive to changes in FabG polynucleotide or polypeptide activity, and binding assays known in the art.

Polypeptides of the invention may be used to identify membrane bound or soluble receptors, if any, for such polypeptide, through standard receptor binding techniques known in the art. These techniques include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (e.g., cells, cell membranes, cell supernatants, tissue extracts, bodily materials). Other methods include biophysical techniques such as surface plasmon resonance and spectroscopy. These screening methods may also be used to identify agonists and antagonists of the polypeptide that compete with the binding of the polypeptide to its receptor(s), if any. Standard methods for conducting such assays are well understood in the art.

The fluorescence polarization value for a fluorescently-tagged molecule depends on the rotational correlation time or tumbling rate. Protein complexes, such as formed by FabG polypeptide associating with another FabG polypeptide or other polypeptide, labeled to comprise a fluorescently-labeled molecule will have higher polarization values than a fluorescently labeled monomeric protein. It is preferred that this method be used to characterize small molecules that disrupt polypeptide complexes.

Fluorescence energy transfer may also be used characterize small molecules that interfere with the formation of FabG polypeptide dimers, trimers, tetramers or higher order structures, or structures formed by FabG polypeptide bound to another polypeptide. FabG polypeptide can be labeled with both a donor and acceptor fluorophore. Upon mixing of the two labeled species and excitation of the donor fluorophore, fluorescence energy transfer can be detected by observing fluorescence of the acceptor. Compounds that block dimerization will inhibit fluorescence energy transfer.

Surface plasmon resonance can be used to monitor the effect of small molecules on FabG polypeptide self-association as well as an association of FabG polypeptide and another polypeptide or small molecule. FabG polypeptide can be coupled to a sensor chip at low site density such that covalently bound molecules will be monomeric. Solution protein can then passed over the FabG polypeptide-coated surface and specific binding can be detected in real-time by monitoring the change in resonance angle caused by a change in local refractive index. This technique can be used to characterize the effect of small molecules on kinetic rates and equilibrium binding constants for FabG polypeptide self-association as well as an association of FabG polypeptide and another polypeptide or small molecule.

A scintillation proximity assay may be used to characterize the interaction between an association of FabG polypeptide with another FabG polypeptide or a different polypeptide. FabG polypeptide can be coupled to a scintillation-filled bead. Addition of radio-labeled FabG polypeptide results in binding where the radioactive source molecule is in close proximity to the scintillation fluid. Thus, signal is emitted upon FabG polypeptide binding and compounds that prevent FabG polypeptide self-association or an association of FabG polypeptide and another polypeptide or small molecule will diminish signal.

In other embodiments of the invention there are provided methods for identifying compounds that bind to or otherwise interact with and inhibit or activate an activity or expression of a polypeptide and/or polynucleotide of the invention comprising: contacting a polypeptide and/or polynucleotide of the invention with a compound to be screened under conditions to permit binding to or other interaction between the compound and the polypeptide and/or polynucleotide to assess the binding to or other interaction with the compound, such binding or interaction preferably being associated with a second component capable of providing a detectable signal in response to the binding or interaction of the polypeptide and/or polynucleotide with the compound; and determining whether the compound binds to or otherwise interacts with and activates or inhibits an activity or expression of the polypeptide and/or polynucleotide by detecting the presence or absence of a signal generated from the binding or interaction of the compound with the polypeptide and/or polynucleotide.

Another example of an assay for FabG agonists is a competitive assay that combines FabG and a potential agonist with FabG-binding molecules, recombinant FabG binding molecules, natural substrates or ligands, or substrate or ligand mimetics, under appropriate conditions for a competitive inhibition assay. FabG can be labeled, such as by radioactivity or a calorimetric compound, such that the number of FabG molecules bound to a binding molecule or converted to product can be determined accurately to assess the effectiveness of the potential antagonist.

It will be readily appreciated by the skilled artisan that a polypeptide and/or polynucleotide of the present invention may also be used in a method for the structure-based design of an agonist or antagonist of the polypeptide and/or polynucleotide, by: (a) determining in the first instance the three-dimensional structure of the polypeptide and/or polynucleotide, or complexes thereof; (b) deducing the three-dimensional structure for the likely reactive site(s), binding site(s) or motif(s) of an agonist or antagonist; (c) synthesizing candidate compounds that are predicted to bind to or react with the deduced binding site(s), reactive site(s), and/or motif(s); and (d) testing whether the candidate compounds are indeed agonists or antagonists.

It will be further appreciated that this will normally be an iterative process, and this iterative process may be performed using automated and computer-controlled steps.

In a further aspect, the present invention provides methods of treating abnormal conditions such as, for instance, a Disease, related to either an excess of, an under-expression of, an elevated activity of, or a decreased activity of FabG polypeptide and/or polynucleotide.

If the expression and/or activity of the polypeptide and/or polynucleotide is in excess, several approaches are available. One approach comprises administering to an individual in need thereof an inhibitor compound (antagonist) as herein described, optionally in combination with a pharmaceutically acceptable carrier, in an amount effective to inhibit the function and/or expression of the polypeptide and/or polynucleotide, such as, for example, by blocking the binding of ligands, substrates, receptors, enzymes, etc., or by inhibiting a second signal, and thereby alleviating the abnormal condition. In another approach, soluble forms of the polypeptides still capable of binding the ligand, substrate, enzymes, receptors, etc. in competition with endogenous polypeptide and/or polynucleotide may be administered. Typical examples of such competitors include fragments of the FabG polypeptide and/or polypeptide.

In still another approach, expression of the gene encoding endogenous FabG polypeptide can be inhibited using expression blocking techniques. This blocking may be targeted against any step in gene expression, but is preferably targeted against transcription and/or translation. An examples of a known technique of this sort involve the use of antisense sequences, either internally generated or separately administered (see, for example, O'Connor, J Neurochem (1991) 56:560 in Oligodeoxynucleotides as Antisense Inhibitors of Gene Expression, CRC Press, Boca Raton, Fla. (1988)). Alternatively, oligonucleotides that form triple helices with the gene can be supplied (see, for example, Lee et al., Nucleic Acids Res (1979) 6:3073; Cooney et al., Science (1988) 241:456; Dervan et al., Science (1991) 251:1360). These oligomers can be administered per se or the relevant oligomers can be expressed in vivo.

Each of the polynucleotide sequences provided herein may be used in the discovery and development of antibacterial compounds. The encoded protein, upon expression, can be used as a target for the screening of antibacterial drugs. Additionally, the polynucleotide sequences encoding the amino terminal regions of the encoded protein or Shine-Delgarno or other translation facilitating sequences of the respective mRNA can be used to construct antisense sequences to control the expression of the coding sequence of interest.

The invention also provides the use of the polypeptide, polynucleotide, agonist or antagonist of the invention to interfere with the initial physical interaction between a pathogen or pathogens and a eukaryotic, preferably mammalian, host responsible for sequelae of infection. In particular, the molecules of the invention may be used: in the prevention of adhesion of bacteria, in particular gram positive and/or gram negative bacteria, to eukaryotic, preferably mammalian, extracellular matrix proteins on in-dwelling devices or to extracellular matrix proteins in wounds; to block bacterial adhesion between eukaryotic, preferably mammalian, extracellular matrix proteins and bacterial FabG proteins that mediate tissue damage and/or; to block the normal progression of pathogenesis in infections initiated other than by the implantation of in-dwelling devices or by other surgical techniques.

In accordance with yet another aspect of the invention, there are provided FabG agonists and antagonists, preferably bacteristatic or bactericidal agonists and antagonists.

The antagonists and agonists of the invention may be employed, for instance, to prevent, inhibit and/or treat diseases.

### GLOSSARY

The following definitions are provided to facilitate understanding of certain terms used frequently herein.

“Bodily material(s) means any material derived from an individual or from an organism infecting, infesting or inhabiting an individual, including but not limited to, cells, tissues and waste, such as, bone, blood, serum, cerebrospinal fluid, semen, saliva, muscle, cartilage, organ tissue, skin, urine, stool or autopsy materials.

“Disease(s)” or “Infection(s)” means (i) bacterial infections, such as staphylococcal infections including, but not limited to infections of the upper respiratory tract (e.g., otitis media, bacterial tracheitis, acute epiglottitis, thyroiditis), the lower respiratory tract (e.g., empyema, lung abscess), the cardiac system (e.g., infective endocarditis), the gastrointestinal tract (e.g., secretory diarrhea, splenic abscess, retroperitoneal abscess), the CNS (e.g., cerebral abscess), eye (e.g., blepharitis, conjunctivitis, keratitis, endophthalmitis, preseptal and orbital cellulitis, darcryocystitis), the kidney or urinary tract (e.g., epididymitis, intrarenal and perinephric abscess, toxic shock syndrome), the skin (e.g., impetigo, folliculitis, cutaneous abscesses, cellulitis, wound infection, bacterial myositis), and the bones and joints (e.g. septic arthritis, osteomyelitis) and/or (ii) an infection caused by or related to a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacterfreundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis.

“Host cell(s)” is a cell that has been introduced (e.g., transformed or transfected) or is capable of introduction (e.g., transformation or transfection) by an exogenous polynucleotide sequence.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following: Algorithm: Needleman and Wunsch, J. Mol Biol. 48: 443-453 (1970)
Comparison matrix: BLOSSUM62 from Hentikoff and Hentikoff, Proc. Natl. Acad. Sci. USA. 89:10915-10919 (1992)
Gap Penalty: 12
Gap Length Penalty: 4
   A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm: Needleman and Wunsch, J. Mol Biol. 48: 443-453 (1970)
Comparison matrix: matches=+10, mismatch=0
Gap Penalty: 50

0Gap Length Penalty: 3

Available as: The “gap” program from Genetics Computer Group, Madison Wis. These are the default parameters for nucleic acid comparisons.

A preferred meaning for “identity” for polypeptides, as the case may be, are provided below.

Polypeptide embodiments further include an isolated polypeptide comprising a polypeptide having at least a 95, 97 or 100% identity to a polypeptide reference sequence of SEQ ID NO:2, wherein said polypeptide sequence may be identical to the reference sequence of SEQ ID NO:2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 by the integer defining the percent identity divided by 100 and then subtracting that product from said total number of amino acids in SEQ ID NO:2, or:
nₐ≦x_{z}−(xₐ·y),
wherein nₐ is the number of amino acid alterations, xₐ is the total number of amino acids in SEQ ID NO:2, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Individual(s)” means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Organism(s)” means a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

“Polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

It will be clear that the invention may be practiced otherwise than as particularly described in the foregoing description and examples.

Numerous modifications and variations of the present invention are possible in light of the above teachings and, therefore, are within the scope of the appended claims.

Each reference cited herein is hereby incorporated by reference in its entirety. Moreover, each patent application to which this application claims priority is hereby incorporated by reference in its entirety.

What is claimed is:

1. An antagonist that inhibits or an agonist that activates an activity a polypeptide selected from the group consisting of: a polypeptide comprising an amino acid sequence which is at least 90% identical to the amino acid sequence of SEQ ID NO:2, and a polypeptide comprising an amino acid sequence as set forth in SEQ ID NO:2, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

2. A method for the treatment of an individual having need to inhibit or activate Fab G polypeptide comprising the steps of: administering to the individual an antibacterially effective amount of an antagonist that inhibits or an agonist that activates an activity of a polypeptide selected from the group consisting of: a polypeptide comprising an amino acid sequence which is at least 90% identical to the amino acid sequence of SEQ ID NO:2, and a polypeptide comprising an amino acid sequence as set forth in SEQ ID NO:2, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

3. A method for the treatment of an individual infected with a bacteria comprising the steps of administering to the individual an antibacterially effective amount of an antagonist that inhibits or an agonist that activates an activity of a polypeptide selected from the group consisting of: a polypeptide comprising an amino acid sequence which is at least 90% identical to the amino acid sequence of SEQ ID NO:2, and a polypeptide comprising an amino acid sequence as set forth in SEQ ID NO:2, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

4. The method of claim 3 wherein said bacteria is selected from the group consisting of a member of the genus Staphylococcus, Staphylococcus aureus, a member of the genus Streptococcus, and Streptococcus pneumoniae.

5. A method for the treatment of an individual having need to inhibit or activate Fab G polypeptide comprising the steps of administering to the individual an antibacterially effective amount of an antagonist that inhibits or an agonist that activates an activity of Fab G selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue (FIG. 5), with an anionic, tetrahedral reaction intermediate (1) being formed, that is potentially charge-stabilized by protonated Lys group prior to proton transfer to form the β-hydroxy-keto product (2).

6. A method for the treatment of an individual infected with a bacteria comprising the steps of administering to the individual an antibacterially effective amount of an antagonist that inhibits or an agonist that activates that activates an activity of Fab G selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

7. The method of claim 6 wherein said bacteria is selected from the group consisting of: a member of the genus Staphylococcus, Staphylococcus aureus, a member of the genus Streptococcus, and Streptococcus pneumoniae.

8. A method for the treatment of an individual infected by Streptococcus pneumoniae comprising the steps of administering to the individual an antibacterially effective amount of an antagonist that inhibits or antagonist that activates an activity of Streptococcus pneumoniae Fab G selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

9. An antagonist that inhibits an activity of a polypeptide selected from the group consisting of: a polypeptide comprising an amino acid sequence which is at least 90% identical to the amino acid sequence of SEQ ID NO:1, and a polypeptide comprising an amino acid sequence as set forth in SEQ ID NO:1, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

10. A method for the treatment of an individual having need to inhibit Fab G polypeptide comprising the steps of administering to the individual an antibacterially effective amount of an antagonist that inhibits an activity of a polypeptide selected from the group consisting of: a polypeptide comprising an amino acid sequence which is at least 90% identical to the amino acid sequence of SEQ ID NO:1, and a polypeptide comprising an amino acid sequence as set forth in SEQ ID NO:1, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen;
hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

11. A method for inhibiting an activity of Fab G polypeptide comprising the steps of contacting a composition comprising said polypeptide with an effective amount of an antagonist that inhibits an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

12. A method for inhibiting an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat} (FIG. 1A); deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer fom Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

13. The method of claim 12 wherein said bacteria is selected from the group consisting of: a member of the genus Staphylococcus, Staphylococcus aureus, a member of the genus Streptococcus, and Streptococcus pneumoniae.

14. A method for inhibiting a growth of bacteria comprising the steps of contacting a composition comprising bacteria with an antibacterially effective amount of an antagonist that inhibits an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

15. The method of claim 14 wherein said bacteria is selected from the group consisting of: a member of the genus Staphylococcus, Staphylococcus aureus, a member of the genus Streptococcus, and Streptococcus pneumoniae.

16. A method for inhibiting a Fab G polypeptide comprising the steps of contacting a composition comprising bacteria with an antibacterially effective amount of an antagonist that inhibits an activity of Fab G, wherein said activity is selected from the group consisting of: NADPH-dependent reduction of acetoacetyl-acyl carrier protein (ACP) to generate β-hydroxyacyl-ACP; deprotonation of a group leading to a diminution in k_{cat}; deprotonation of a general acid responsible for donating a proton to the carbonyl oxygen during its reduction; binding of an ionizable group putatively binding the pyrophosphate bridge of NADPH; catalysis involving a lysine residue as a general acid; a conformational change inducing formation a low-barrier hydrogen bond (LBHB) in ketone reduction mechanism; a conformational change upon acetoacetyl-CoA binding resulting in formation of an LBHB; a conformational change upon acetoacetyl-CoA binding resulting in formation of an LBHB between Tyr157 and Lys161; energy provided from Tyr157 and Lys161; energy from forming an LBHB between Tyr157 and Lys161 facilitating proton transfer from Lys157 to the carbonyl oxygen; proton transfer from Lys157 to carbonyl oxygen; formation of an LBHB between Tyr157 and an Asp residue; strengthening of the role of Tyr157 in facilitating general acid catalysis; strengthening of the role of Tyr157 in facilitating general acid catalysis by Lys161; compression of active site; compression of active site resulting in the formation of a LBHB facilitating proton transfer to the carbonyl oxygen; hydride transfer from NADPH proceeding proton transfer from the Lys residue; formation of an anionic, tetrahedral reaction intermediate; formation of a charge-stabilized intermediate by protonated Lys group prior to proton transfer to form the β-hydroxy-keto product (2); and hydride transfer from NADPH proceeding proton transfer from the Lys residue, with an anionic, tetrahedral reaction intermediate (1) being formed, that is potentially charge-stabilized by protonated Lys group prior to proton transfer to form the β-hydroxy-keto product (2).

17. The method of claim 16 wherein said bacteria is selected from the group consisting of: a member of the genus Staphylococcus, Staphylococcus aureus, a member of the genus Streptococcus, and Streptococcus pneumoniae.


SEQUENCE LISTING (2 sequences)

SEQ ID NO: 1 - protein, 243 aa, Streptococcus pneumoniae
MKLEHKNIFITGSSRGIGLAIAHKFAQAGANIVLNSRGAISEELLAEFSNYGIKVVPISGDVSDFADAKRMIDQAIAELGSVDVLVNNAGITQDTLMLKMTEADFEKVLKVNLTGAFNMTQSVLKPMMKAREGAIINMSSVVGLMGNIGQANYAASKAGLIGFTKSVAREVASRNIRVNVIAPGMIESDMTAILSDKIKEATLAQIPMKEFGQAEQVADLTVFLAGQDYLTGQVIAIDGGLSM

SEQ ID NO: 2 - DNA, 732 bp, Streptococcus pneumoniae
atgaaactagaacataaaaatatctttattacaggttcgagtcgtggaattggtcttgccatcgcccacaagtttgctcaagcaggagccaacattgtcttaaacagtcgtggggcaatctcagaagaattgctcgctgagttttcaaactatggtatcaaggtggttcccatttcaggagatgtatcagattttgcagacgctaagcgtatgattgatcaagctattgcagaactgggttcagtagatgttttggtcaacaatgcagggattacccaagatactcttatgctcaagatgacagaagcagattttgaaaaagtgctcaaggtcaatctgactggtgcctttaatatgacacaatcagtcttgaaaccgatgatgaaagccagagaaggtgctatcattaatatgtctagtgttgttggtttgatggggaatattggtcaagctaactatgctgcttctaaggctggcttgattggctttaccaagtctgtggcacgcgaggtcgctagtcggaatatacgagtcaatgtgattgctccaggaatgattgagtctgatatgacagctatcttatcagataagattaaggaagctacactagctcagattccgatgaaagaatttgggcaggcagagcaggttgcagatttgacagtatttttagcaggccaagattatctaactggtcaagtgattgccattgatggtggcttaagtatgtag